(19) United States
(12) Patent Application Publication
(10) Pub. No.: US 2015/0126892 A1
(43) Pub. Date: May 7, 2015

(54) CLOUD SERVER FOR PROCESSING ELECTROENCEPHALGRAPHY INFORMATION, AND APPARATUS FOR PROCESSING ELECTROENCEPHALOGRAPHY INFORMATION BASED ON CLOUD SERVER

(71) Applicant: ELECTRONICS AND TELECOMMUNICATIONS RESEARCH INSTITUTE, Daejeon (KR)

(72) Inventors: Kwi Hoon KIM, Daejeon (KR); Myung Ae CHUNG, Daejeon (KR); Woong Shik YOU, Sejong (KR)

(73) Assignee: ELECTRONICS AND TELECOMMUNICATIONS RESEARCH INSTITUTE, Daejeon (KR)

(21) Appl. No.: 14/242,365

(22) Filed: Apr. 1, 2014

(30) Foreign Application Priority Data
Nov. 4, 2013 (KR) 10-2013-0132824

(51) Int. Cl. A61B 5/00; A61B 5/16; A61B 5/0476

(52) U.S. Cl. CPC A61B 5/0006; A61B 5/0476; A61B 5/168
USPC 600/545; 600/544

(57) ABSTRACT

Disclosed are a cloud server for processing electroencephalography information, and an apparatus for processing electroencephalography information which measures, transmits, or processes electroencephalography information based on the cloud server. The cloud server includes: a communication unit communicatively connected with a wireless network to receive electroencephalography information of a user; and a electroencephalography information processing unit configured to analyze a mental state of the user from the received electroencephalography information, and provide a command or information corresponding to a result of the analysis as a processing result, in which the processing result is provided to the user or another user through the wireless network.

### CROSS-REFERENCE TO RELATED APPLICATION

This application is based on and claims priority from Korean Patent Application No. 10-2013-0132824, filed on Nov. 4, 2013, with the Korean Intellectual Property Office, the disclosure of which is incorporated herein in its entirety by reference.

### BACKGROUND

1. Field

The present invention relates to an apparatus for processing electroencephalography information, and more particularly to a cloud server for processing electroencephalography information, and an apparatus for processing electroencephalography information which measures, transmits, or processes electroencephalography information based on the cloud server.

2. Discussion of Related Art

Electroencephalography (EEG), which is one of bio-signals for temporarily and spatially recognizing an activity of a brain, is widely utilized in a clinic and research in a brain function. A method of measuring a current generated according to an activity of a brain by attaching an electrode to a scalp of a user is commonly used for measuring electroencephalographys. A waveform of a electroencephalography signal may be considerably changed according to a portion of a head, to which the electrode is attached, or thinking or a mental state of a user. In general, electroencephalographys have a size of several uV to several hundreds of uV, and a frequency of the electroencephalographys is generable from 0 to a very large region, but in general, it is known that when the frequency of the electroencephalography is 0 to 50 Hz, a many distinguishing meanings are included in the electroencephalographys. The types of electroencephalographys for each frequency band and mental states of a person according to the types of electroencephalographys are illustrated in FIG. 1. Referring to FIG. 1, a waveform has a frequency band width of 8 to 12 Hz, and is a waveform of electroencephalographys which are easily generated when a normal adult is in a stable mental state and generally observed in an occipital region and a parietal region. In the meantime, β waveform has a frequency band width of 13 to 30 Hz, and is a waveform of electroencephalographys which are easily generated when a person concentrates his/her mind or a brain activity is increased and observed in a parietal region and a temporal region.

As described above, a person generates different electroencephalographys according to a region of a brain, an arousal state, and a degree of psychological stability, so that research on various devices using a electroencephalography generation characteristic has been conducted. Particularly, an interest in a Brain-Computer Interface (BCI) system, which allows a user to communicate with a computer through a thinking of the user without a separate input device, has been increased.

However, the BCI has a problem in that electroencephalography processing equipment measuring and processing electroencephalographys is generally excessively large and heavy, and when the electroencephalography processing equipment is simplified in order to solve the problem, there is a limit in that various performances including precision deteriorates.

### SUMMARY

The present invention has been made in an effort to provide a cloud apparatus for processing electroencephalography information, and an apparatus for processing electroencephalography information, which achieves lightness and improved precision, based on a cloud system, and a method of processing electroencephalography information based on a cloud system.

Further, the present invention has been made in an effort to provide an apparatus and a method of processing electroencephalography information, which control an electronic device with electroencephalography information generated by a user based on a cloud server or a cloud system processing electroencephalography information.

Further, the present invention has been made in an effort to provide an apparatus and a method of processing electroencephalography information, which define a predetermined electroencephalography information pattern based on a cloud system, and receive expression of user's intention through the defined electroencephalography information pattern.

Further, the present invention has been made in an effort to provide an apparatus and a method of processing electroencephalography information, which analyze electroencephalography information collected from a plurality of subjects based on a cloud system, and selectively provide different contents.

An embodiment of the present invention provides a cloud server, including: a communication unit communicatively connected with a wireless network to receive electroencephalography information of a user; and a electroencephalography information processing unit configured to analyze a mental state of the user from the received electroencephalography information, and provide a command or information corresponding to a result of the analysis as a processing result, in which the processing result is provided to the user or another user through the wireless network.

Another embodiment of the present invention provides an apparatus for processing electroencephalography information, including: a measurement unit configured to measure electroencephalography information of a electroencephalography generator; and a transmitter connected with the measurement unit, and configured to transmit the measured electroencephalography information to the outside in order to provide the measured electroencephalography information to a cloud server, in which the cloud server analyzes a mental state of the electroencephalography generator from the measured electroencephalography information, and provides a command or information corresponding to a result of the analysis as a processing result.

Yet another embodiment of the present invention provides an apparatus for processing electroencephalography information, including: a receiver configured to receive a processing result from a cloud server; and a driver configured to drive at least one predetermined function according to the received processing result, in which the cloud server receives electroencephalography information of a user, analyzes a mental state of the user from the received electroencephalography information, and provides a command or information corresponding to a result of the analysis as a processing result.

According to the exemplary embodiments of the present invention, electroencephalography information of a user is measured and processed based on a cloud system, so that the apparatus for processing electroencephalography information may become light and precision may be improved, and an electronic device may be controlled with electroencephalography information generated by a user.

Further, according to the exemplary embodiments of the present invention, it is possible to efficiently process electroencephalography information by receiving expression of an intention of a user through a predefined electroencephalography information pattern or perform an operation according to the received expression of the intention based on the cloud system, and selectively provide various contents in accordance with behaviors, tendencies, or preferences of subjects by analyzing electroencephalography information collected from a plurality of subjects.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The above and other features and advantages of the present invention will become more apparent to those of ordinary skill in the art by describing in detail embodiments thereof with reference to the attached drawings in which:

FIG. 1 is a table illustrating the types of electroencephalographys for each frequency band and mental states of a person according to the types of electroencephalographys;

FIG. 2 is a diagram schematically illustrating a electroencephalography information processing system based on a cloud according to an exemplary embodiment of the present invention;

FIG. 3 is an exemplary diagram specifically illustrating a user device illustrated in FIG. 2;

FIG. 4 is an exemplary block diagram specifically illustrating a cloud server illustrated in FIG. 2;

FIG. 5 is a block diagram specifically illustrating a electroencephalography information processing system according to an exemplary embodiment of the present invention;

FIG. 6 is a flowchart illustrating a electroencephalography information processing method of the electroencephalography information processing system illustrated in FIG. 5;

FIG. 7 is a block diagram specifically illustrating a electroencephalography information processing system according to another exemplary embodiment of the present invention;

FIG. 8 is a flowchart illustrating a electroencephalography information processing method of the electroencephalography information processing system illustrated in FIG. 7;

FIG. 9 is a pattern table defining patterns of electroencephalography information according to exemplary embodiments of the present invention;

FIG. 10 is a block diagram illustrating a electroencephalography information processing system processing electroencephalography information by using a predefined electroencephalography information pattern according to the exemplary embodiments of the present invention;

FIG. 11 is a flowchart illustrating a electroencephalography information processing method of the electroencephalography information processing system illustrated in FIG. 10;

FIG. 12 is a block diagram illustrating a electroencephalography information processing system processing a electroencephalography information group collected from a plurality of users according to the exemplary embodiments of the present invention; and

FIG. 13 is a flowchart illustrating a electroencephalography information processing method of the electroencephalography information processing system illustrated in FIG. 12.

### DETAILED DESCRIPTION

Hereinafter, an embodiment of the present invention will be described with reference to the accompanying drawings in detail. However, the present invention is not limited to an embodiment disclosed below and may be implemented in various forms and the scope of the present invention is not limited to the following embodiments. Rather, the embodiment is provided to more sincerely and fully disclose the present invention and to completely transfer the spirit of the present invention to those skilled in the art to which the present invention pertains, and the scope of the present invention should be understood by the claims of the present invention.

Throughout this specification and the claims that follow, when it is described that an element is “coupled” to another element, the element may be “directly coupled” to the other element or “electrically coupled” to the other element through a third element. Throughout the specification and the claims, unless explicitly described to the contrary, the word “comprise” and variations such as “comprises” or “comprising” will be understood to imply the inclusion of stated elements but not the exclusion of any other elements.

FIG. 2 is a diagram schematically illustrating a electroencephalography information processing system based on a cloud according to an exemplary embodiment of the present invention. Referring to FIG. 2, a electroencephalography information processing system 1000 includes a user device 1100, a cloud server 1200, and a wireless network 1300.

The user device 1100 is connected with the cloud server 1200 through the wireless network 1300 to transmit data to the cloud server 1200 or receive data from the cloud server 1200. The user device 1100 measures electroencephalographys generated from a user or a electroencephalography generator (for example, a person or an animal generating electroencephalographys), and transmits the measured electroencephalographys or information derived from the measured electroencephalographys to the cloud server 1200 as electroencephalography information.

Otherwise, the user device 1100 receives a processing result generated by processing the electroencephalography information by the cloud server 1200, and performs a specific operation according to the received processing result, or display or store the processing result. In this case, the electroencephalography information processed by the cloud server 1200 may be received from the user device 1100, or may be received from another user device (not shown) different from the user device 1100.

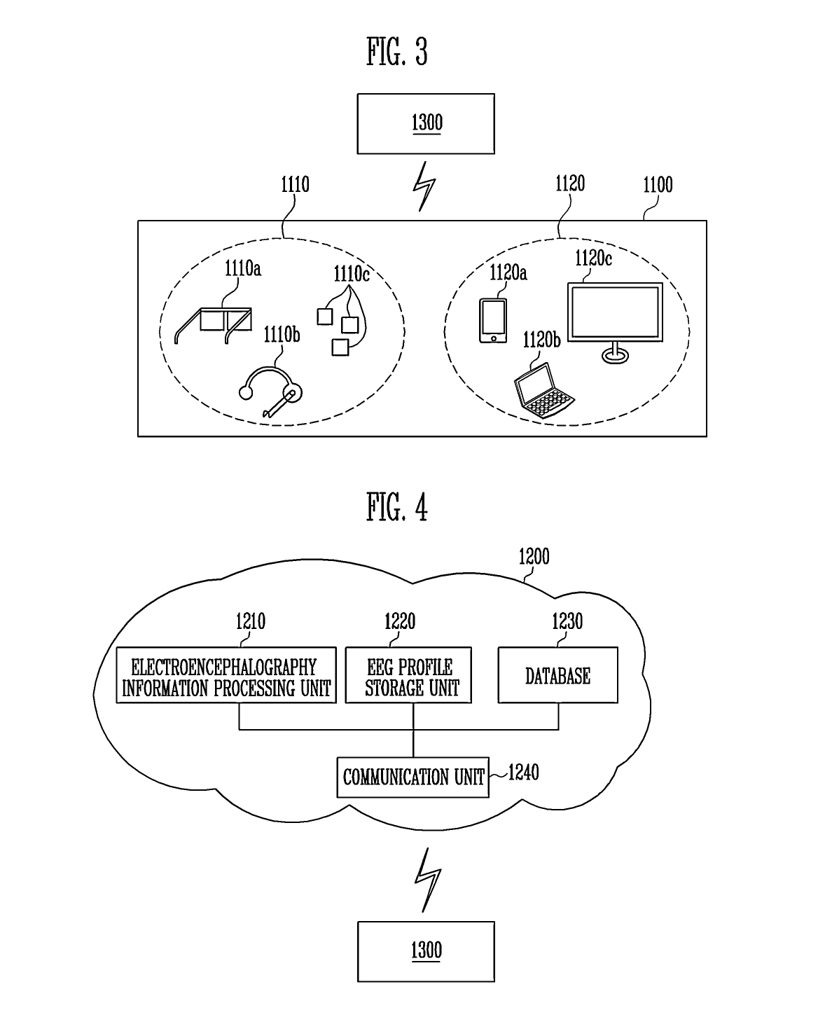

A particular configuration, function, and operating method of the user device 1100 will be described in more detail with reference to FIG. 3 below.

The cloud server 1200 is communicatively connected with a plurality of communication devices including the user device 1100 through the wireless network 1300. The cloud server 1200 provides communication devices connected with the cloud server 1200 with a general or individualized service (for example, a electroencephalography information processing service) by using autonomously including processing means. As an exemplary embodiment, the cloud server 1200 may include a log-on means individually or integrally controlling access of the communication devices.

The cloud server 1200 receives electroencephalography information from the user device 1100 or another user device (not shown) communicatively connected with the cloud server 1200. The cloud server 1200 analyzes a type, a pattern, and a meaning of the electroencephalography information or information derived from the received electroencephalography information, and generates a command or information corresponding to an analysis result. The generated command or information is transmitted to the user device 1100 as a processing result. As an exemplary embodiment, the electroencephalography information received by the cloud server 1200 or the processing result generated by the cloud server 1200 may be stored in a separate storage unit (not shown) included inside the cloud server 1200.

As an exemplary embodiment, the cloud server 1200 may group electroencephalography information collected from a plurality of user devices, and analyze meaningful group information (for example, a group mental state or expression of a plurality of intentions of the group) from the grouped electroencephalography information. In this case, the cloud server 1200 may adopt statistical means (for example, a regression method and a correlation coefficient analysis method) widely known in the art for analyzing the group information. Further, the cloud server 1200 may generate a command or information corresponding to the analysis result and transmit the generated command or information to the user device 1100.

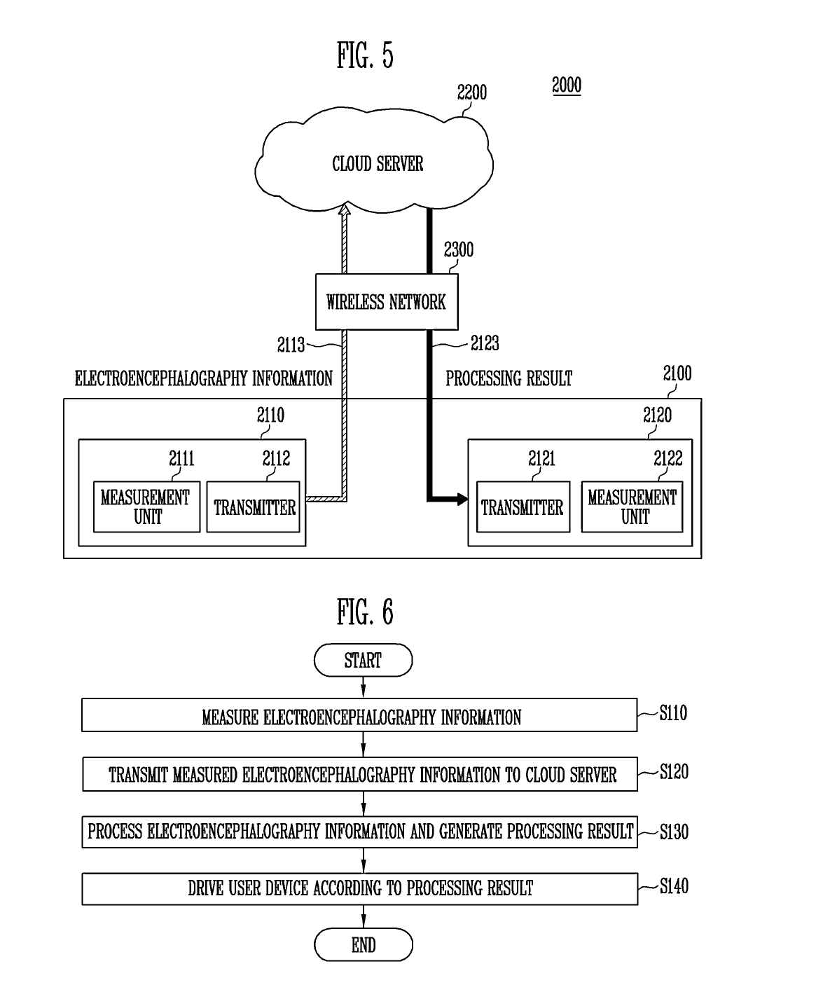

A particular configuration, function, and operating method of the cloud server 1200 will be described in more detail with reference to FIG. 4 below.

The wireless network 1300 communicatively connects the user device 1100 and the cloud server 1200. The wireless network 1300 relays data transmission between the user device 1100 and the cloud server 1200 through, for example, a mobile communication network 1310 or a wireless LAN 1320 provided by a mobile communication company. The wireless network 1300 may include hardware or software optimized to various communication standards, and include a general communication means enabling an object to network with another object.

The wireless network 1300 may include one or more communication means selected from the group including a wireless Local Area Network (LAN), a Metropolitan Area Network (MAN), a Global System for Mobile Network (GSM), an Enhanced Data GSM Environment (EDGE), High Speed Downlink Packet Access (HSDPA), Wideband Code Division Multiple Access (W-CDMA), Code Division Multiple Access (CDMA), Time Division Multiple Access (TDMA), Bluetooth, Zigbee, Wi-Fi, a Voice over Internet Protocol (VoIP), LTE Advanced, IEEE802.16m, WirelessMAN-Advanced, HSPA+, 3GPP Long Term Evolution (LTE), Mobile WiMAX (IEEE 802.16e), UMB (formerly EV-DO Rev. C), Flash-OFDM, iBurst and MBWA (IEEE 802.20) systems, HIPERMAN, Beam-Division Multiple Access (BDMA), World Interoperability for Microwave Access (Wi-MAX), and ultrasonic waves utilizing communication, but is not limited thereto.

According to the aforementioned configuration, the cloud server 1200 processes the measured electroencephalography information instead of the user device 1100. Otherwise, the cloud server 1200 provides a command or information corresponding to the processing result of the electroencephalography information to the user device 1100. Accordingly, the user device 1100 does not need to include a means for analyzing and processing the electroencephalography information, so that the user device 1100 may become lighter and a manufacturing cost thereof may be decreased.

Further, the cloud server 1200 may include a centralized high performance processing device, thereby more effectively and precisely performing a electroencephalography information processing function than the individual user device 1100. Accordingly, performance and precision of a electroencephalography information processing method may be improved.

Further, the cloud server 1200 may be communicatively connected with a plurality of users to collect electroencephalography information from the plurality of users, analyze the collected electroencephalography information, and selectively provide various contents in accordance with behaviors, tendencies, or preferences of the users.

FIG. 3 is an exemplary diagram specifically illustrating the user device illustrated in FIG. 2. The user device 1100 communicates with the cloud server 1200 (see FIG. 2) through the wireless network 1300. Referring to FIG. 3, the user device 1100 includes a measurement device 1110 measuring electroencephalographys or a terminal device 1120 performing a specific function according to the processing result of the electroencephalography information.

However, it is illustrated that both the measurement device 1110 and the terminal device 1120 are included within the user device 1100, which, however, does not mean that the measurement device 1110 and the terminal device 1120 are included together. The user device 1100 includes any one of the measurement device 1110 and the terminal device 1120, or includes all of the measurement device 1110 and the terminal device 1120 as separate hardwares. Otherwise, the user device 1100 may be formed in a form in which the measurement device 1110 and the terminal device 1120 are integrated to be embedded in one hardware.

The measurement device 1110 measures electroencephalographys from a electroencephalography generator. Here, the electroencephalography generator, which is a subject generating electroencephalographys, generally corresponds to a person or an animal. In a case where the electroencephalography generator is a person, the measurement device 1110 may be configured as a device for detecting and measuring generation of electroencephalographys while being in contact with a head of a person.

Referring to FIG. 3, an example of the measurement device 1110 includes an glasses-type measurement device 1110a, a headset-type measurement device 1110b, and a speaker-type measurement device 1110c. Each of the measurement devices 1110a, 1110b, and 1110c includes a measurement unit (not shown) measuring electroencephalography information generated from a head of a person. Here, the electroencephalography information is commonly called electroencephalographys or data indicating electroencephalographys. As an exemplary embodiment, the measurement unit may be formed in an electrode form attached to a head of a person. In this case, the measurement unit may include at least one reference electrode (not shown) providing a reference voltage and at least one detection electrode (not shown) detecting a waveform of electroencephalographys. Each of the reference electrode and the detection electrode are included in the measurement devices 1110a, 1110b, and 1110c as a part of the measurement devices 1110a, 1110b, and 1110c.

Each of the measurement devices 1110a, 1110b, and 1110c includes a transmitter (not shown) transmitting the measured electroencephalography information (or electroencephalographys) to the cloud server 1200, the terminal device 1120, or another user device (not shown) or an interface unit (not shown). Detailed contents of the transmitter and the interface unit will be described in more detail with reference to FIG. 5.

In the meantime, it is illustrated herein that an example of the measurement device 1110 includes the glasses-type measurement device 1110a, the headset-type measurement device 1110b, and the speaker-type measurement device 1110c, but the form of the measurement device 1110 is not limited thereto. The measurement device 1110 may be formed of any one of or a combination of the measurement devices 1110a, 1110b, and 1110c, and any device configured to measure electroencephalographys from the electroencephalography generator may be used as the measurement device 1110.

The terminal device 1120 receives the processing result generated by the cloud server 1200. In this case, the processing result may include a computer readable command or information directing an operation of the terminal device 1120.

The terminal device 1120 may perform a specific function according to the received processing result, re-transmit the processing result to another device, or store the processing result in an internal memory. For example, the terminal device 1120 may display information (or a still image or a video related to the processing result) included in the processing result, or perform at least one of functions of the terminal device 1120 (for example, alarm, call, application execution, data communication with another device, storage of data in the internal memory, deletion of data stored in the internal memory, or reproduction of music or a video) according to the command included in the processing result.

Referring to FIG. 3, an example of the terminal device 1120 includes a smart phone 1120a, a notebook computer 1120b, and a TV 1120c. Each of the terminal devices 1120s includes a receiver (not shown) receiving the processing result provided by the cloud server through the wireless network 1300. Further, each of the terminal devices 1120s includes a driver (not shown) which is communicatively connected with the receiver to perform at least one function of the terminal device 1120. The driver may be a calculation module including a Central Processing Unit (CPU) or an Application Processing unit (AP) of the terminal device 1120.

As an exemplary embodiment, each of the terminal devices 1120a, 1120b, and 1120c may further include an interface unit (not shown) for receiving the electroencephalography information provided by the measurement device 1110. In this case, each of the terminal devices 1120a, 1120b, and 1120c may further include a transmitter (not shown) for re-transmitting the electroencephalography information received through the interface unit to the cloud server 1200. Detailed contents of the interface unit and the transmitter included in each of the terminal devices 1120a, 1120b, and 1120c will be described in more detail with reference to FIG. 7.

In the meantime, here, the smart phone 1120a, the notebook computer 1120b, and the TV 1120c are illustrated as an example of the terminal device 1120, but the form of the terminal device 1120 is not limited thereto. The terminal device 1120 may be formed of any one of or a combination of the terminal devices 1120a, 1120b, and 1120c, and any device configured to perform a specific function according to the processing result provided by the cloud server 1200 may be used as the terminal device 1120.

FIG. 4 is an exemplary block diagram specifically illustrating the cloud server illustrated in FIG. 2. The cloud server 1200 receives electroencephalography information or transmits a processing result of the electroencephalography information through the wireless network 1300. Referring to FIG. 4, the cloud server 1200 includes a electroencephalography information processing unit 1210, an EEG profile storage unit 1220, a database 1230, and a communication unit 1240.

The electroencephalography information processing unit 1210 analyzes electroencephalography information received through the wireless network 1300, and generates or provides a command or information corresponding to the analysis result as a processing result. For example, the electroencephalography information processing unit 1210 may read a mental state of a user (or a electroencephalography generator generating electroencephalography information) from the electroencephalography information, and generate information indicating the read metal condition or generate a command corresponding to the read mental state. For example, when the mental state read from the electroencephalography information is a state of tension, the electroencephalography information processing unit 1210 may generate image data or sound data as information indicating the mental state. Otherwise, when the mental state read from the electroencephalography information is a state of tension, the electroencephalography information processing unit 1210 may generate a computer readable command so that the user device 1110 (see FIG. 2) plays music having a relaxation effect in order to relax the state of tension.

As an exemplary embodiment, the electroencephalography information processing unit 1210 may statistically analyze a plurality of elements of electroencephalography information through statistic means, which are well known in the art, and generate a command or information corresponding to a result of the statistical analysis as a processing result.

As an exemplary embodiment, the electroencephalography information processing unit 1210 may read profile information from the EEG profile storage unit 1220 and utilize the read profile information in the analysis of the electroencephalography information in order to decrease an analysis error of the received information. Electroencephalographys generated by a person (or a electroencephalography generator) may have a deviation, so that a waveform of the generated electroencephalography or a electroencephalography generation position may be minutely different depending on a person even though the people have the same mental state. The profile information includes individual information about a electroencephalography tendency, an individual electroencephalography type, or an individual electroencephalography deviation, and the electroencephalography information processing unit 1210 may decrease an analysis error of the electroencephalography information by referring to the profile information during the analysis of the electroencephalography information. More detailed contents of the EEG profile storage unit 1220 and the profile information will be described below.

The EEG profile storage unit 1220 stores the profile information. The profile information includes personal profile information about an individual electroencephalography deviation as described above. According to the profile information, frequency bands, which the cloud server 1200 recognizes as a specific waveform (for example, a waveform), may be different according to an individual deviation. Further, according to the profile information, the cloud server 1200 may analyze that people, in which electroencephalography information of the same frequency is measured, are in the different mental states.

In the meantime, the profile information may include reference profile information referred in a general electroencephalography analysis, in addition to information about the individual electroencephalography deviation. The reference profile information includes information about electroencephalography tendencies, electroencephalography types, or electroencephalography deviations of general people. The cloud server 1200 may precisely analyze the received electroencephalography signal in a customization type by selectively referring to the personal profile information or the reference profile information among the profile information.

As an exemplary embodiment, the profile information may be generated by the electroencephalography information processing unit 1210 by referring to the analysis results of the received electroencephalography information. In this case, the electroencephalography information processing unit 1210 may recognize an individual electroencephalography tendency, type, or deviation through a plurality of number of times of analysis for an individual person, and generate the profile information based on the recognized individual electroencephalography tendency, type, or deviation.

As an exemplary embodiment, the profile information may be generated by referring to Sensory Evoked Potentials (SEP) and Event-Related Potentials (ERP) of the electroencephalography generator. The SEP generally means electroencephalography signals in an unconscious state generated when a person responds to stimulation. For example, the SEP means electroencephalography potentials induced through different senses, such as visually induced potentials, or tactually induced or auditorily induced potentials. The ERP means electroencephalographys induced in connection with a specific accident. The SEP and the ERP are measured and transmitted by the measurement device 1110 (see FIG. 3) to be referred in order to generate the profile information.

The database 1230 is a storage medium capable of temporarily or permanently storing data, and provides a storage means to the cloud server 1200. It is illustrated that the database 1230 is one module, but is not limited thereto, and the database 1230 may include a plurality of memories or storage media. Further, the database 1230 may include heterogeneous memories or storage media together. The cloud server 1200 may store temporal data generated during the processing of the electroencephalography signal or data generated as the processing result of the electroencephalography signals in the database 1230.

The communication unit 1240 performs communication or data transception between the cloud server 1200 and the wireless network 1300. The communication unit 1240 includes an interface means communicatively connected to the wireless network 1300, and receives electroencephalography information from the wireless network 1300. The received electroencephalography information may be provided to each of the modules 1210, 1220, and 1230 of the cloud server 1200. Otherwise, the communication unit 1240 transmits the result of the electroencephalography information proceed by the cloud server 1200 to the wireless network 1300. The processing results transmitted to the wireless network 1300 are re-transmitted to the user device 1100 (see FIG. 2).

FIG. 5 is a block diagram specifically illustrating a electroencephalography information processing system according to an exemplary embodiment of the present invention. Referring to FIG. 5, a cloud server 2200 of a electroencephalography information processing system 2000 receives electroencephalography information from a measurement device 2110 through a wireless network 2300 (2113), and provides a terminal device 2120 with a processing result of the received electroencephalography information through the wireless network 2300 (2123).

In the present exemplary embodiment, the measurement device 2110 measures electroencephalography information from a electroencephalography generator through a measurement unit 2111. The measurement unit 2111 may be formed of a contact/non-contact type detecting means for detecting electroencephalographys of the generator. The measured electroencephalography information is transmitted to a transmitter 2112, and the transmitter 2112 transmits the transmitted electroencephalography information to the cloud server 2200 through the wireless network 2300. In this case, the measurement device 2110 may perform a specific authentication procedure through a log-on means of the cloud server 2200 for data communication with the cloud server 2200.

The cloud server 2200 analyzes the received electroencephalography information, and generates the processing result through a series of electroencephalography information processing operation of generating a command or information corresponding to the analysis result. Detailed contents of the method of generating the processing result by the cloud server 2200 and the generated processing result are substantially the same as those in the above description. The cloud server 2200 transmits the generated processing result to the terminal device 2120 through the wireless network 2300.

The terminal device 2120 may perform a specific function according to the received processing result, re-transmit the processing result to another device, or store the processing result in an internal memory. For example, the terminal device 2120 may display information (or a still image or a video related to the processing result) included in the processing result, or perform at least one of functions of the terminal device 2120 (for example, alarm, call, application execution, data communication with another device, storage of data in the internal memory, deletion of data stored in the internal memory, or reproduction of music or a video) according to the command included in the processing result.

According to the aforementioned configuration, the cloud server 2200 processes the measured electroencephalography information instead of the user device 2100, and provides the user device 2100 with a command or information corresponding to the processing result of the electroencephalography information. Accordingly, the user device 2100 does not need to include a means for analyzing and processing the electroencephalography information, so that the user device 2100 may become lighter and a manufacturing cost thereof may be decreased.

Further, the cloud server 2200 may include a centralized high performance processing means, thereby more effectively and precisely performing a electroencephalography information processing function than the individual user device 2100. Accordingly, performance and precision of a electroencephalography information processing method may be improved.

In the meantime, other particular contents of the user device 2100, the cloud server 2200, the wireless network 2300, the measurement device 2110, and the terminal device 2120, which are not described herein, are substantially the same as the aforementioned contents of the user device 1100, the cloud server 1200, the wireless network 1300, the measurement device 1110, and the terminal device 1120.

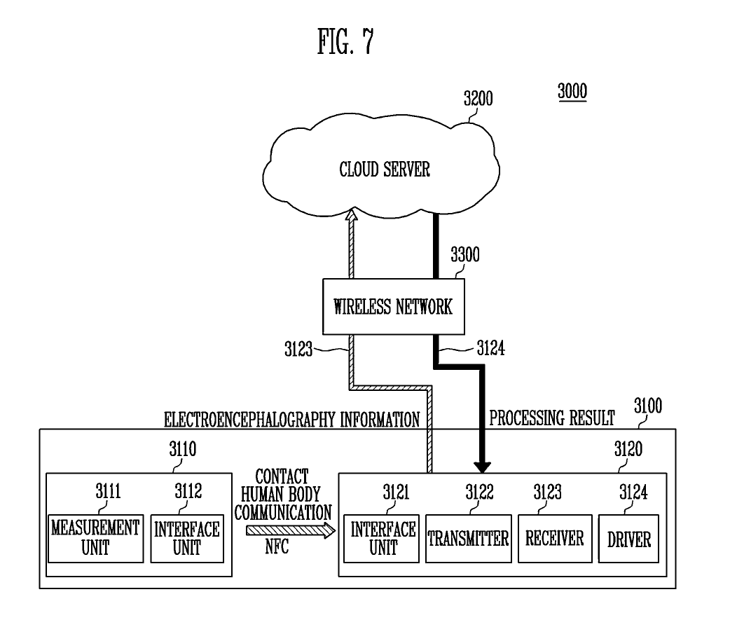

FIG. 6 is a flowchart illustrating a electroencephalography information processing method of the electroencephalography information processing system illustrated in FIG. 5. Referring to FIG. 6, the electroencephalography information processing method includes step S110 to step S140.

In step S110, the measurement device 2110 measures electroencephalography information from a electroencephalography generator. In this case, the electroencephalography information may be measured through a contact type or a non-contact type detecting means, such as a detecting electrode, included in the measurement device 2110. The measured electroencephalography information is transmitted to the transmitter 2112 capable of communicating with the wireless network 2300.

In step S120, the measurement device 2110 transmits the measured electroencephalography information to the cloud server 2200 through the wireless network 2300. Here, the wireless network 2300 relaying communication between the measurement device 2110 and the cloud server 2200 may include various wireless communication means, such as a mobile communication network or a wireless LAN provided by a mobile communication company.

As an exemplary embodiment, the measurement device 2110 may perform an authentication procedure through a specific log-on means for accessing the cloud server 2200.

In step 130, the cloud server 2200 analyzes the received electroencephalography information, and generates a processing result through a series of electroencephalography information processing operation of generating a command or information corresponding to the analysis result. The generated processing result is transmitted to the terminal device 2120 through the wireless network 2300.

In step S140, the terminal device 2120 may perform a function included in the terminal device 2120 according to the received processing result, re-transmit the processing result to another device, or store the processing result in an internal memory.

FIG. 7 is a block diagram specifically illustrating a electroencephalography information processing system according to another exemplary embodiment of the present invention. Referring to FIG. 7, in a electroencephalography information processing system 3000, a measurement device 3110 measures electroencephalography information and transmits the measured electroencephalography information to a terminal device 3120, and the terminal device 3120 transmits the electroencephalography information through a wireless network 3300 to a cloud server 3200 again (3123). Further, the cloud server 3200 processes the received electroencephalography information, and provides the terminal device 3120 with the processing result of the electroencephalography information to the terminal device 3120 through the wireless network 3300 (3124).

In the present exemplary embodiment, the measurement device 3110 measures electroencephalography information from a electroencephalography generator through a measurement unit 3111. The measurement unit 3111 may be formed of a contact/non-contact type detecting means for detecting electroencephalographys of the generator. The measured electroencephalography information is transmitted to the terminal device 3120 via interface units 3112 and 3121.

Each of the interface units 3112 and 3121 may be a module including an active signal transmitting means, or a medium or media (for example, the interface unit 3112 is a frame forming a physical structure of the measurement device 3110 or a composition filled in at least a part of the physical structure of the measurement device 3110) passively and simply transmitting vibration, an electrical signal, and the like.

The electroencephalography information transmission between the interface units 3112 and 3121 may be performed through a skin contact of a user, human body communication, or Near Field Communication (NFC). For example, the electroencephalography information may be transmitted from the interface unit 3112 to the other interface unit 3121 through a contact of grasping the terminal device 3120 with a hand by a user. Otherwise, the electroencephalography information transmission between the interface units 3112 and 3121 may be performed through human body communication.

The human body communication is a communication method of transmitting data through a human body by using a current flowing in the human body, and has an advantage of low power and high speed communication. Detailed contents of the human body communication are widely known in the art, so that descriptions thereof will be omitted. When the human body communication is used, even though the terminal device 3120 is not in direct contact with a skin of the user (for example, in a case where the terminal device 3120 is inside a handbag or a pocket of trousers), the electroencephalography information may be transmitted from the measurement device 3110 to the terminal device 3120. Otherwise, electroencephalography information may be transmitted through NFC between the interface units 3112 and 3121.

The terminal device 3120 transmits the received electroencephalography information to the cloud server 3200 through a transmitter 3122. In this case, the transmission of the electroencephalography information is performed via the wireless network 3300, and the terminal device 3120 may perform a specific authentication procedure through a log-on means of the cloud server 3200 for data communication with the cloud server 3200.

The cloud server 3200 analyzes the received electroencephalography information, and generates the processing result through a series of electroencephalography information processing operation of generating a command or information corresponding to the analysis result. Detailed contents of the method of generating the processing result by the cloud server 3200 and the generated processing result are substantially the same as those in the above description. The cloud server 3200 transmits the generated processing result to the terminal device 3120 through the wireless network 3300.

The terminal device 3120 may perform a specific function according to the received processing result, re-transmit the processing result to another device, or store the processing result in an internal memory. For example, the terminal device 3120 may display information (or a still image or a video related to the processing result) included in the processing result, or perform at least one of functions of the terminal device 3120 (for example, alarm, call, application execution, data communication with another device, storage of data in the internal memory, deletion of data stored in the internal memory, or reproduction of music or a video) according to the command included in the processing result.

According to the aforementioned configuration, the cloud server 3200 processes the measured electroencephalography information instead of the user device 3100, and provides the user device 3100 with a command or information corresponding to the processing result of the electroencephalography information. Accordingly, the user device 3100 does not need to include a means for analyzing and processing the electroencephalography information, so that the user device 3100 may become lighter and a manufacturing cost thereof may be decreased.

In the meantime, in the present exemplary embodiment, a transmitter 3122 for transmitting the electroencephalography information to the cloud server 3200 is included in the terminal device 3120 instead of the measurement device 3110, and thus the measurement device 3110 may become lighter. The measurement device 3110 is generally attached to the body (particularly, a head) of the user, so that the lightness of the measurement device 3110 may be effective in improving convenience of the user.

Further, in the present exemplary embodiment, the transmitter 3122 is removed from the measurement device 3110, so that an electromagnetic wave may be far spaced apart from the human body (particularly, the head of the user). When the transmitter 3122 transmits the electroencephalography information toward the cloud server 3200, the transmitter 3122 may generate comparatively strong electromagnetic waves. It is obvious that strong electromagnetic waves are harmful to a human body, so that the configuration in which the transmitter 3122 is included in the terminal device 3120 relatively spaced far from the human body of the user (particularly, the head) may contribute to convenience and health protection of the user.

Further, the cloud server 3200 may include a centralized high performance processing means, thereby more effectively and precisely performing a electroencephalography information processing function than the individual user device 3100. Accordingly, performance and precision of a electroencephalography information processing method may be improved.

In the meantime, other particular contents of the user device 3100, the cloud server 3200, the wireless network 3300, the measurement device 3110, and the terminal device 3120, which are not described herein, are substantially the same as the aforementioned contents of the user device 1100, the cloud server 1200, the wireless network 1300, the measurement device 1110, and the terminal device 1120.

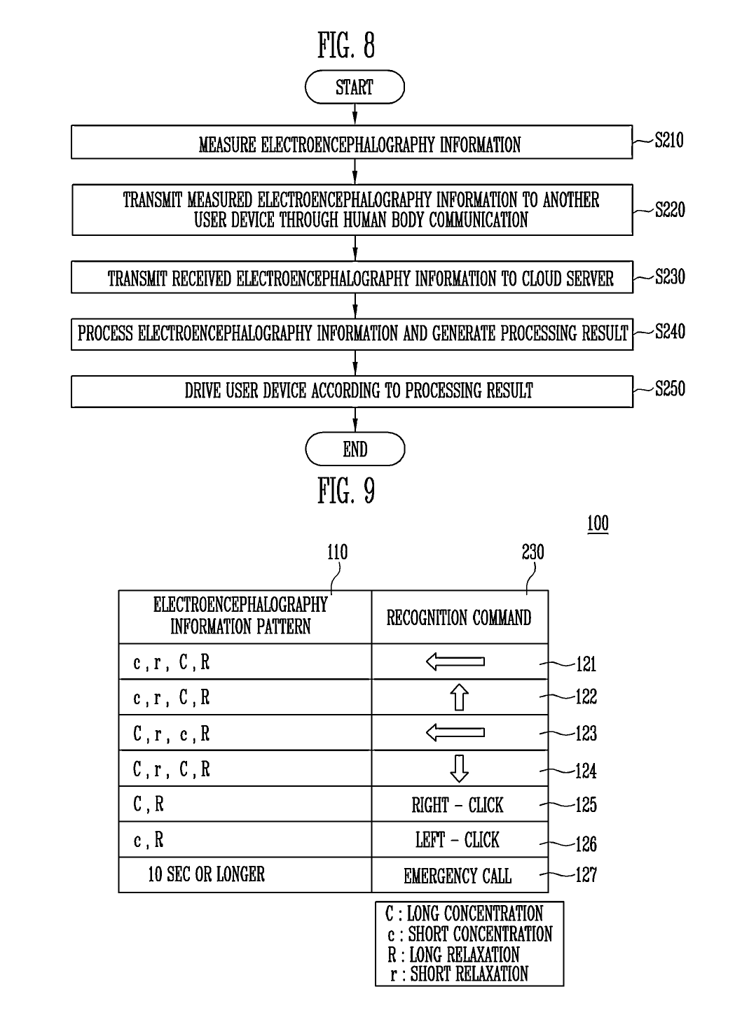

FIG. 8 is a flowchart illustrating a electroencephalography information processing method of the electroencephalography information processing system illustrated in FIG. 7. Referring to FIG. 8, the electroencephalography information processing method includes step S210 to step S250.

In step S210, the measurement device 3110 measures electroencephalography information from a electroencephalography generator. In this case, the electroencephalography information may be measured through a contact type or a non-contact type detecting means, such as a detecting electrode, included in the measurement device 3110. The measured electroencephalography information is transmitted to the interface unit 3112 within the measurement device 3110.

In step S220, the electroencephalography information is transmitted from the measurement device 3110 to the terminal device 3120 through the interface units 3112 and 3121. The electroencephalography information is transmitted to the terminal device 3120 by directly contacting the measurement device 3110 to the terminal device 3120 by the user wearing the measurement device 3110 or by using human body communication or NFC. Detailed contents for the electroencephalography information transmission between the measurement device 3110 and the terminal device 3120 are the same as those described with reference to FIG. 7.

In step S230, the terminal device 3120 transmits the measured electroencephalography information to the cloud server 3200 through the wireless network 3300. Here, the wireless network 3300 relaying communication between the terminal device 3120 and the cloud server 3200 may include various wireless communication means, such as a mobile communication network or a wireless LAN provided by a mobile communication company.

As an exemplary embodiment, the terminal device 3120 may perform an authentication procedure through a specific log-on means for accessing the cloud server 3200.

In step 240, the cloud server 3200 analyzes the received electroencephalography information, and generates a processing result through a series of electroencephalography information processing operation of generating a command or information corresponding to the analysis result. The generated processing result is transmitted to the terminal device 3120 through the wireless network 3300.

In step S250, the terminal device 3120 may perform a function included in the terminal device 3120 according to the received processing result, re-transmit the processing result to another device, or store the processing result in an internal memory.

FIG. 9 is a pattern table defining patterns of electroencephalography information according to the exemplary embodiments of the present invention.

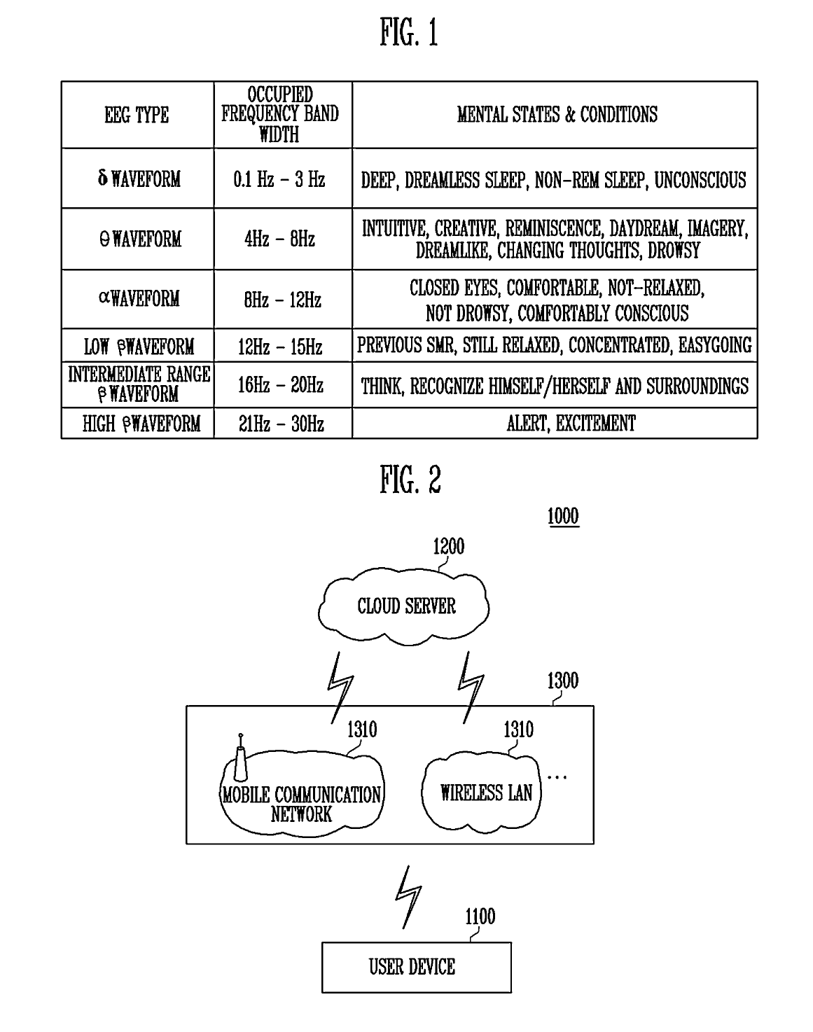

The pattern table 100 is referred in order to determine a recognition command corresponding to a received electroencephalography information pattern by comparing the received electroencephalography information pattern with a defined electroencephalography information pattern. The user intentionally forms a electroencephalography information pattern formed of repeated mind concentration and relaxation, and the electroencephalography information pattern formed by the user is transmitted to the cloud server 1200 (see FIG. 1). When the received electroencephalography information pattern is matched with the pre-defined electroencephalography information pattern, the cloud server 1200 interprets that a command (recognition command) corresponding to the pre-defined electroencephalography information pattern is a command intended by the user. Further, the cloud server 1200 transmits the processing result including the recognition command to the user device 1100 (see FIG. 1) so that the user device 1100 performs an operation according to the recognition command.

Referring to FIG. 9, a pattern table 100 defining the electroencephalography information patterns includes a electroencephalography information pattern field 110 and a recognition command field 120.

The electroencephalography information pattern field 110 represents pre-defined electroencephalography information patterns. The electroencephalography information pattern in the electroencephalography information pattern field 110 is formed of a combination of long concentration C, short concentration c, long relaxation R, and short relaxation r.

The recognition command field 120 represents commands (recognition commands) corresponding to the electroencephalography information patterns defined in the electroencephalography information pattern field 110. For example, the first electroencephalography information pattern of the electroencephalography information pattern field 110 corresponds to the first recognition command of the recognition command field 120. Similarly, the n^{th} electroencephalography information pattern of the electroencephalography information pattern field 110 corresponds to the n^{th} recognition command of the recognition command field 120.

When the user forms a specific electroencephalography information pattern through concentration and relaxation, the cloud server 1200 compares the electroencephalography information pattern of the user with the electroencephalography information pattern defined in the electroencephalography information pattern field 110. As a result of the comparison, when the electroencephalography information pattern of the user is matched with the electroencephalography information pattern defined in the electroencephalography information pattern field 110, the cloud server 1200 recognizes that the electroencephalography information of the user validly means a specific command and determines that the corresponding recognition command is a command intended by the user. The determined recognition command is transmitted to the user device 1100 as a processing result of the electroencephalography information pattern so that the user device 1100 performs an operation according to the recognition command.

For example, when the electroencephalography information pattern formed by the user is a combination of short concentration, short relaxation, short concentration, and long relaxation (c, r, c, R), the cloud server 1200 determines that the user intends a left arrow command 121, and transmits a processing result including the left arrow command 121 to the user device 1100. The user device 1100 performs an operation (for example, an operation of moving a cursor on a displayed image to the left) corresponding to the left arrow command 121 of the processing result.

Similarly, when the electroencephalography information pattern formed by the user is a combination of short concentration, short relaxation, long concentration, and long relaxation (c, r, C, R), the cloud server 1200 determines that the user intends an up arrow command 122, and transmits a processing result including the up arrow command 122 to the user device 1100. The user device 1100 performs an operation (for example, an operation of moving a cursor on a displayed image one line above) corresponding to the up arrow command 122 of the processing result.

As described above, the cloud server 1200 matches the different electroencephalography information patterns to the different recognition commands 121, 122, 123, 124, 125, 126, and 127, respectively, thereby recognizing the electroencephalography information pattern formed by the user as a specific user command.

As an exemplary embodiment, the electroencephalography information pattern may be formed of any one of concentration and relaxation, as well as the combination of concentration and relaxation. For example, when the user continuously concentrates his/her mind for 10 seconds or longer to form the electroencephalography information pattern (long C), the cloud server 1200 recognizes the formed electroencephalography information pattern (long C) as a user electroencephalography information pattern intending an emergency call. Further, the cloud server 1200 transmits the processing result including the command for emergency call to the user device 1100. The user device 1100 makes an emergency call to a family member, a security company, a police, or a fire station in response to the emergency call command.

Here, it is exemplified that the electroencephalography information pattern is defined through the combination of the electroencephalographys divided into two types (concentration and relaxation), such as Morse code, and duration time of the electroencephalographys. However, this is illustrative, and the method of forming or defining the electroencephalography information pattern is not limited thereto. The electroencephalography information pattern may be formed by using different electroencephalography forms, not concentration and relaxation, and any method capable of specifying and defining the patterns of the electroencephalographys may be used for defining the electroencephalography information pattern mentioned herein.

According to the aforementioned configuration, the user may express his/her intention or command by intentionally forming the electroencephalography information pattern. Accordingly, the user may control the user device 1100 in a desired manner only through thinking without a separate operation.

FIG. 10 is a block diagram illustrating a electroencephalography information processing system processing electroencephalography information by using a predefined electroencephalography information pattern according to the exemplary embodiments of the present invention. Referring to FIG. 10, a cloud server 4200 of a electroencephalography information processing system 4000 receives a electroencephalography information pattern from a user device 4100 through a wireless network 4300 (4110), and provides a user device 4100 with a processing result of the received electroencephalography information pattern through a wireless network 4300 (4120). The user device of FIG. 10 may have the same configuration as that of any one of the user devices 1100, 2100, and 3100 of FIGS. 2, 5, and 7.

When the user forms a specific electroencephalography information pattern through concentration and relaxation, the user device 4100 measures the formed electroencephalography information pattern and transmits the measured electroencephalography information pattern to the cloud server 4200. In this case, the electroencephalography information pattern may be transmitted to the cloud server 4200 via the wireless network 4300. The cloud server 4200 compares the received electroencephalography information pattern with electroencephalography information patterns defined in a pattern table 4210. Here, the pattern table 4210 is substantially the same as the pattern table 100 described with reference to FIG. 9, and is stored in a storage medium (for example, the database 1230 (see FIG. 4)) inside the cloud server 4200. When the electroencephalography information pattern of the user is matched with the electroencephalography information pattern defined in the pattern table 4210, the cloud server 4200 transmits a processing result including the corresponding command to the user device 4100. The user device 4100 performs the command corresponding to the electroencephalography information pattern according to the received processing result.

According to the aforementioned configuration, the cloud server 4200 processes the measured electroencephalography information pattern instead of the user device 4100. Otherwise, the cloud server 4200 provides the user device 4100 with the command corresponding to the processing result of the electroencephalography information pattern. Accordingly, the user device 4100 does not need to include a means for analyzing and processing the electroencephalography information pattern, so that the user device 4100 may become lighter and a manufacturing cost thereof may be decreased.

Further, the cloud server 4200 may include a centralized high performance processing means, thereby more effectively and precisely performing a electroencephalography information processing function than the individual user device 4100. Accordingly, the electroencephalography information pattern may be more precisely and rapidly processed.

Further, the user may express his/her intention or command by intentionally forming the electroencephalography information pattern. Accordingly, the user may control the user device 4100 in a desired manner only through thinking without a separate operation.

In the meantime, other particular contents of the user device 4100, the cloud server 4200, the wireless network 4300, the measurement device 4110, and the terminal device 4120, which are not described herein, are substantially the same as the aforementioned contents of the user device 1100, the cloud server 1200, the wireless network 1300, the measurement device 1110, and the terminal device 1120.

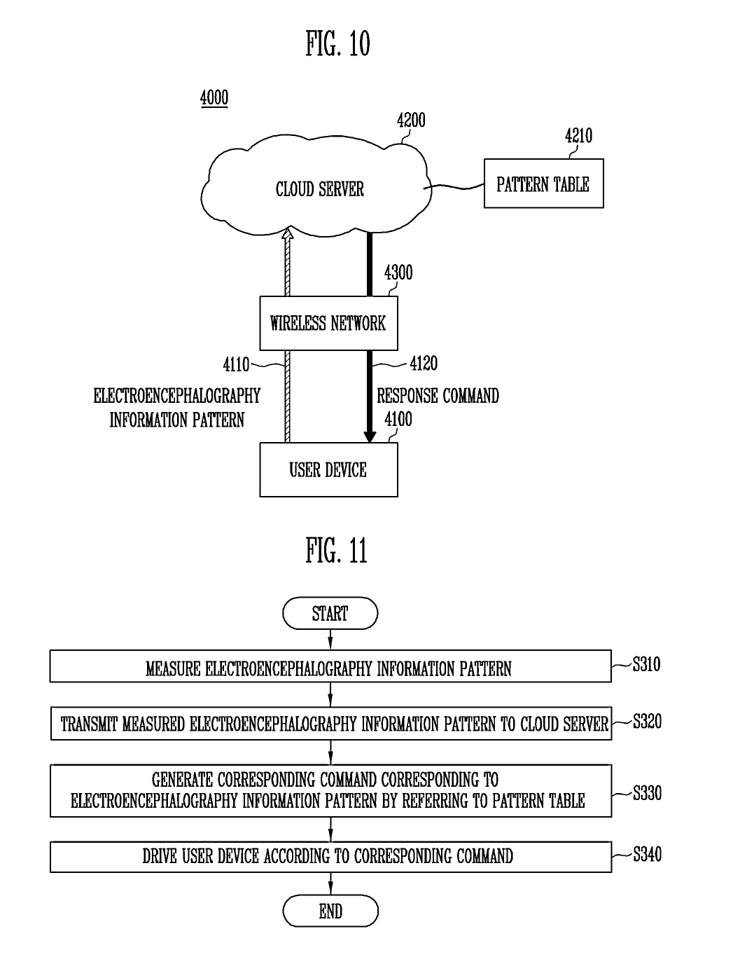

FIG. 11 is a flowchart illustrating a electroencephalography information processing method of the electroencephalography information processing system illustrated in FIG. 10. Referring to FIG. 11, the electroencephalography information processing method includes step S310 to step S340.

In step S310, the user device 4100 measures a electroencephalography information pattern generated from a electroencephalography generator. In this case, the electroencephalography information pattern may be measured through a contact type or a non-contact type detecting means, such as a detecting electrode.

In step S320, the user device 4100 transmits the measured electroencephalography information pattern to the cloud server 4200 through the wireless network 4300. Here, the wireless network 4300 relaying communication between the user device 4100 and the cloud server 4200 may include various wireless communication means, such as a mobile communication network or a wireless LAN provided by a mobile communication company.

As an exemplary embodiment, the user device 4100 may perform an authentication procedure through a specific log-on means for accessing the cloud server 4200.

In step S330, the cloud server 4200 compares the received electroencephalography information pattern with a pre-defined electroencephalography information pattern by referring to the pattern table. Further, the cloud server 4200 generates a response command (or recognition command) responding to the received electroencephalography information pattern according to a result of the comparison. The generated response command is transmitted to the user device 4100 through the wireless network 4300 as a processing result.

In step S440, the user device 4100 performs a function included in the user device 4100 according to the response command received as the processing result.

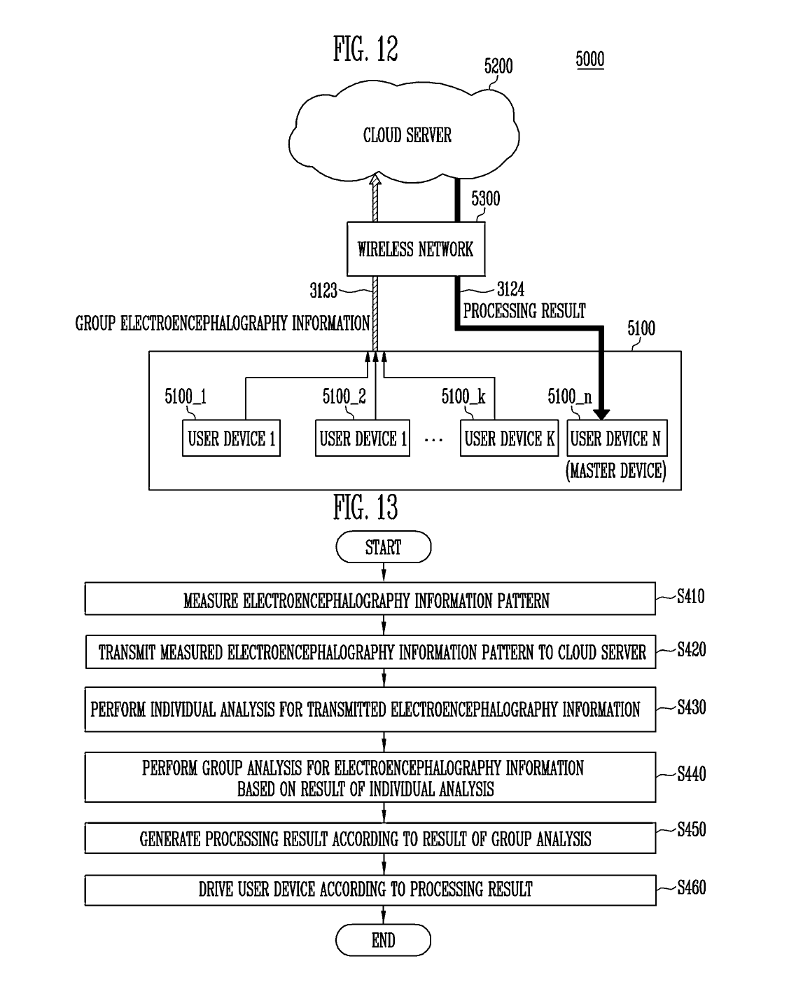

FIG. 12 is a block diagram illustrating a electroencephalography information processing system processing a electroencephalography information group collected from a plurality of users according to the exemplary embodiments of the present invention. Referring to FIG. 12, a cloud server 5200 of a electroencephalography information processing system 5000 receives electroencephalography information from a plurality of user devices 5100_1, 5100_2, . . . , and 5100_{—}k through a wireless network 5300 (5110), and provides another user device 5100n with a processing result of the received electroencephalography information through the wireless network 5300.

A user device group 5100 includes the plurality of user devices 5100_1, 5100_2, . . . , 5100_{—}k, and 5100_{—}n providing the electroencephalography information or receiving the processing results. Each of the user devices 5100_1, 5100_2, . . . , 5100_{—}k, and 5100_{—}n of FIG. 12 may have the same configuration as that of any one of the user devices 1100, 2100, and 3100 of FIGS. 2, 5, and 7.

In the present exemplary embodiment, the cloud server 5200 receives electroencephalography information of the plurality of users, individually determines a meaning of each of the electroencephalography information by analyzing the received electroencephalography information, and then a grouped mental state of the plurality of users as a whole based on a result of the individual determination. Here, the group analysis means to synthetically analyze tendencies, conditions, preferences, and the like of the users based on the electroencephalography information of the plurality of users, of which the electroencephalography information is measured, as a whole.

As an exemplary embodiment, the cloud server 5200 may periodically receive the electroencephalography information from the users, and periodically perform the group analysis on the electroencephalography information of the users.

As an exemplary embodiment, the cloud server 5200 may use statistical means (for example, an average value calculating method, a dispersion calculating method, a percentage calculating method, and a regression analysis method) well known in the art for the group analysis.

Hereinafter, particular exemplary embodiments in which the electroencephalography information of the users is processed through the aforementioned group analysis will be described.

First, the electroencephalography information processing system 5000 may be utilized in a teaching method of teaching students. In this case, the user devices 5100_1, 5100_2, . . . , and 5100_{—}k are allocated to the students, respectively, to measure electroencephalography information of the students. As an exemplary embodiment, the user devices 5100_1, 5100_2, . . . , and 5100_{—}k may be formed in headsets or glasses wearing on heads of the students. The electroencephalography information of the students measured by the user devices 5100_1, 5100_2, . . . , and 5100_{—}k is transmitted to the cloud server 5200 as group electroencephalography information.

The cloud server 5200 individually analyzes the received electroencephalography information of the students, and analyzes a mental state of each of the students. Further, the cloud server 5200 determines a general tendency, concentration level, concern level, or mental state of the students based on the analyzed mental state of the students, and transmits a result of the determination to a master device 5100n, which a teacher wears, as a processing result.

For example, the cloud server 5200 analyzes the electroencephalography information of the students, and determines a current individual concentration condition of each of the students for a class. Further, based on the individual concentration condition, the cloud server 5200 determines a general concentration level (for example, a percentage of the students concentrating on the class) or a general concern level of the students. As a result of the determination, the cloud server 5200 transmits the determination result as the processing result as it is, or transmits additional information derived from the determination result (for example, a notice message warning that a ratio of the students, who do not concentrate on the class, exceeds a predetermined value) together with the determination result as the processing result. The teacher may more effectively perform the class by referring to the processing result transmitted to the master device 5100n.

In the meantime, it is described here that the cloud server 5200 transmits the processing result only to the master device 5100n, but the present invention is not limited thereto. The cloud server 5200 may independently and separately transmit the processing result even to the user devices 5100_1, 5100_2, . . . , and 5100_{—}k providing the electroencephalography information. For example, the cloud server 5200 may transmit the processing result displaying a current concentration condition of the students to the user devices 5100_1, 5100_2, . . . , and 5100_{—}k. Accordingly, when the students concentrates on the class, green light may be turned on in the user devices (for example, a headset) which the students wear, and when the students do not concentrate on the class, red light may be turned on in the user devices. When red light of many user devices is turned on, the teacher may see the user devices of the students and attempt to change a teaching method, like changing the class to have contents arousing more interest.

Next, the electroencephalography information processing system 5000 may be utilized in a method of analyzing an advertisement effect. In this case, the electroencephalography information processing system 5000 may be utilized in order to pre-analyze an expected effect of an advertisement or analyze an effect of an executed advertisement ex post facto.

For example, an advertiser creates various versions of advertisements and gives a trial performance to customers. In this case, the user devices 5100_1, 5100_2, . . . , and 5100_{—}k are allocated to the customers, respectively, to measure electroencephalography information of the customers. As an exemplary embodiment, the user devices 5100_1, 5100_2, . . . , and 5100_{—}k may be formed in headsets or glasses wearing on heads of the customers. The electroencephalography information of the customers measured by the user devices 5100_1, 5100_2, . . . , and 5100_{—}k is transmitted to the cloud server 5200 as group electroencephalography information.

The cloud server 5200 individually analyzes the received electroencephalography information about the customers, and determines concentration levels, concern levels, tension levels, or relaxation levels when the customers watch the advertisement. Further, the cloud server 5200 determines a general advertisement response rate of the customers based on the determined concentration levels, concern levels, tension levels, or relaxation levels of the customers, and transmits a result of the determination to the master device 5100n possessed by the advertiser as a processing result. In this case, the processing result may simply include response rates of the customers according to each version of the advertisements, include determination information for determining the most effective advertisement according to the response rates of the customers, and additional information indicating a specific moment of the advertisement of a specific version having the highest response rate of the customers.

Next, the electroencephalography information processing system 5000 may be utilized in social mind broadcasting. The social mind broadcasting means broadcasting contents expected to have a high response rate of the viewers by estimating responses of the viewers for contents to be broadcasted in advance. For example, the electroencephalography information processing system 5000 may estimate a response of a viewer for expected conclusions of a drama and broadcast the drama with a conclusion desired by a plurality of viewers.

For the social mind broadcasting, the cloud server 5200 receives a response of a viewer for a specific part in broadcasting contents as electroencephalography information. In this case, the user devices 5100_1, 5100_2, . . . , and 5100_{—}k are allocated to the viewers, respectively, to measure electroencephalography information of the viewers. As an exemplary embodiment, the user devices 5100_1, 5100_2, . . . , and 5100_{—}k may be formed in headsets or glasses wearing on heads of the viewers. The electroencephalography information of the viewers measured by the user devices 5100_1, 5100_2, . . . , and 5100_{—}k is transmitted to the cloud server 5200 as group electroencephalography information.

The cloud server 5200 individually analyzes the received electroencephalography information of the viewers and determines mental states (for example, tension levels, relaxation levels, preference levels, or aversion levels) when the viewers watch a specific part of the broadcasting. Further, based on the determined mental states of the viewers, the cloud server 5200 determines a general scenario preference level of the viewers, and transmits a result of the determination to the master device 5100n possessed by a broadcasting provider (or a broadcasting station) as a processing result.

For example, when the general concentration level or preference level of the viewers is high at a part at which A appears in contents of the broadcasting as a result of the performance of the group analysis based on the mental states of the viewers, the cloud server 5200 determines that the viewers want a conclusion in which A becomes happy. The cloud server 5200 transmits a conclusion according to the group analysis to the master device 5100n as a processing result, and the broadcasting provider may refers the transmitted processing result in order to determine contents to be broadcasted.

In the meantime, it is described here that the user devices 5100_1, 5100_2, . . . , and 5100_{—}k transmitting the electroencephalography information is distinguished from the user device 5100n (master device) receiving the processing result, but the exemplary embodiments of the present invention are not limited thereto. As illustrated, the master device 5100n may be a separate device distinguished from the user devices 5100_1, 5100_2, . . . , and 5100_{—}k and be one of the user devices 5100_1, 5100_2, . . . , and 5100_{—}k, or all of the user devices 5100_1, 5100_2, . . . , and 5100_{—}k may be the master devices receiving the processing result.

According to the aforementioned configuration, the cloud server 5200 may be communicatively connected with a plurality of users to collect electroencephalography information from the plurality of users, individually analyze and group-analyze the collected electroencephalography information, and selectively provide various contents in accordance with behaviors, tendencies, or preferences of the users.

In the meantime, other particular contents of user devices 5100_1, 5100_2, . . . , and 5100_{—}k, the cloud server 5200, and the wireless network 5300, which are not described herein, are substantially the same as the aforementioned contents of the user device 1100, the cloud server 1200, and the wireless network 1300.

FIG. 13 is a flowchart illustrating a electroencephalography information processing method of the electroencephalography information processing system illustrated in FIG. 12. Referring to FIG. 13, the electroencephalography information processing method includes steps S410 to S460.

In step S410, the plurality of user devices 5100_1, 5100_2, . . . , and 5100_{—}k measures electroencephalography information from the viewers to which the plurality of user devices 5100_1, 5100_2, . . . , and 5100_{—}k is allocated. In this case, the electroencephalography information may be measured through a contact type or a non-contact type detecting means, such as a detecting electrode.

In step S420, the plurality of user devices 5100_1, 5100_2, . . . , and 5100_{—}k transmits the electroencephalography information measured through the 5300 to the cloud server 5200 as group electroencephalography information. Here, the wireless network 5300 relaying communication between the plurality of user devices 5100_1, 5100_2, . . . , and 5100_{—}k and the cloud server 5200 may include various wireless communication means, such as a mobile communication network or a wireless LAN provided by a mobile communication company.

As an exemplary embodiment, the user devices 5100_1, 5100_2, . . . , and 5100_{—}k may perform an authentication procedure through a specific log-on means for accessing the cloud server 5200.

In step S430, the cloud server 5200 performs an individual analysis on the received electroencephalography information, and individually analyzes the mental states of the users. A detailed method of performing the individual analysis by the cloud server 5200 is the same as that described with reference to FIG. 12.

In step S440, the cloud server 5200 performs the group analysis on the received electroencephalography information based on a result of the individual analysis. The cloud server 5200 may use statistical means well known in the art in the group analysis. A detailed method of performing the group analysis by the cloud server 5200 is the same as that described with reference to FIG. 12.

In step S450, the cloud server 5200 generates a processing result according to the result of the group analysis and transmits the generated result to the user device 5100_{—}n.

In step S460, the user device 5100_{—}n may display the result of the group analysis, notify the result of the group analysis through another display means, such as a sound, re-transmit the result of the group analysis to another device, store the result of the group analysis in an internal memory, or provide a user with the result of the group analysis by other various methods according to the processing result.

As described above, the embodiment has been disclosed in the drawings and the specification. The specific terms used herein are for purposes of illustration, and do not limit the scope of the present invention defined in the claims. Accordingly, those skilled in the art will appreciate that various modifications and another equivalent example may be made without departing from the scope and spirit of the present disclosure. Therefore, the sole technical protection scope of the present invention will be defined by the technical spirit of the accompanying claims.

What is claimed is:

1. A cloud server, comprising:
a communication unit communicatively connected with a wireless network to receive electroencephalography information of a user; and
a electroencephalography information processing unit configured to analyze a mental state of the user from the received electroencephalography information, and provide a command or information corresponding to a result of the analysis as a processing result,
wherein the processing result is provided to the user or another user through the wireless network.

2. The cloud server of claim 1, further comprising:
an electroencephalography profile storage unit configured to store profile information indicating electroencephalography tendencies, electroencephalography types, or electroencephalography deviations of general people,
wherein the electroencephalography information processing unit analyzes the mental state of the user by referring to the profile information.

3. The cloud server of claim 1, further comprising:
an EEG profile storage unit configured to store profile information indicating an individual electroencephalography tendency, electroencephalography type, or electroencephalography deviation of the user,
wherein the electroencephalography information processing unit analyzes the mental state of the user by referring to the profile information.

4. The cloud server of claim 1, wherein the electroencephalography information includes a electroencephalography information pattern formed by combining one or more electroencephalographys, and
the electroencephalography information processing unit compares the electroencephalography information pattern with a predefined electroencephalography information pattern, and selectively provides a command corresponding to the predefined electroencephalography information pattern as the processing result according to a result of the comparison.

5. The cloud server of claim 4, wherein the electroencephalography information pattern is changed according to a waveform or a duration time of the one or more electroencephalographys.

6. The cloud server of claim 1, wherein the electroencephalography information processing unit individually analyzes a mental state of each of a plurality of users from electroencephalography information of the plurality of users, performs a group analysis for determining a grouped mental state of the plurality of users based on a result of the individual analysis, and provides a command or information corresponding to a result of the group analysis as the processing result.

7. The cloud server of claim 6, wherein the plurality of users are students receiving the same class, and
the result of the group analysis includes information indicating concentration levels or concern levels of the plurality of users for the class.

8. The cloud server of claim 6, wherein the plurality of users are customers watching the same advertisement, and
the result of the group analysis includes information indicating concentration levels or concern levels of the plurality of users for at least a part of the advertisement.

9. The cloud server of claim 6, wherein the plurality of users are viewers watching the same broadcasting, and
the result of the group analysis includes information indicating concentration levels or concern levels of the plurality of users for at least a part of the broadcasting.

10. An apparatus for processing electroencephalography information, comprising:
a measurement unit configured to measure electroencephalography information of a electroencephalography generator; and
a transmitter connected with the measurement unit, and configured to transmit the measured electroencephalography information to the outside in order to provide the measured electroencephalography information to a cloud server,
wherein the cloud server analyzes a mental state of the electroencephalography generator from the measured electroencephalography information, and provides a command or information corresponding to a result of the analysis as a processing result.

11. The apparatus of claim 10, wherein the transmitter transmits the measured electroencephalography information to the cloud server through a wireless network.

12. The apparatus of claim 11, further comprising:
a driver configured to receive the processing result provided by the cloud server, and drive at least one predetermined function according to the processing result.

13. The apparatus of claim 10, wherein the transmitter transmits the measured electroencephalography information to a user device connected with the apparatus for processing the electroencephalography information, and
the user device transmits the received electroencephalography information to the cloud server through a wireless network.

14. The apparatus of claim 13, wherein the apparatus for processing the electroencephalography information is communicatively connected with the user device through a skin contact of a user using the apparatus for processing the electroencephalography information, human body communication, or near field wireless communication.

15. An apparatus for processing electroencephalography information, comprising:
a receiver configured to receive a processing result from a cloud server; and
a driver configured to drive at least one predetermined function according to the received processing result,
wherein the cloud server receives electroencephalography information of a user, analyzes a mental state of the user from the received electroencephalography information, and provides a command or information corresponding to a result of the analysis as a processing result.
